(19) United States
(12) Patent Application Publication
(10) Pub. No.: US 2004/0260380 A1
(43) Pub. Date: Dec. 23, 2004

(54) Devices for delivering multiple stenting structures in situ

(75) Inventors: Doron Marco, Tel-Aviv (IL); Tuvia Dror Kutscher, Shoham (IL)

Correspondence Address: MORRISON & FOERSTER LLP, 755 PAGE MILL RD, PALO ALTO, CA, 94304-1018, US

(73) Assignee: D-Crown LTD, Tel Aviv (IL)

(21) Appl. No.: 10/636,877

(22) Filed: Aug. 6, 2003

Related U.S. Application Data
(60) Provisional application No. 60/480,690, filed on Jun. 18, 2003.
(60) Provisional application No. 60/486,096, filed on Jul. 7, 2003.
(60) Provisional application No. 60/491,243, filed on Jul. 31, 2003.

(51) Int. Cl. A61F 2/06

(52) U.S. Cl. 623/1.11

(57) ABSTRACT

Described here are devices and methods for delivering and deploying multiple prostheses having stenting properties within body lumens. More specifically, stent precursor structures, delivery assemblies, and methods for delivering and deploying stent precursor members to form stenting structures in situ, at a selected target site within a body lumen are described. In some variations, the stent precursor members are made of super-elastic materials, in other variations, the stent precursor members are made of plastic materials. The stent precursor member may optionally be loaded with, coated by, or otherwise made to release a biologically active agent. The stent precursor structure may optionally include a guide member to support the stent precursor members. In some variations, the guide member is made of super-elastic materials or plastic materials. Devices and methods for delivering multiple stent precursor members to form multiple stenting structures sequentially and simultaneously are also described.

### CROSS-REFERENCE TO RELATED APPLICATIONS

This application claims priority to U.S. Provisional Application Ser. Nos. 60/480,690, 60/______, 60/______, which were filed on Jun. 18, 2003, Jul. 25, 2003, and Jul. 29, 2003, respectively, each of which is hereby incorporated by reference in its entirety.

### FIELD

Described here are devices and methods for delivering, deploying, and forming prostheses having stenting properties within body lumens. More specifically, stent precursor structures, delivery assemblies, and methods for delivering and deploying stent precursor members to form stenting structures at a selected target site within a body lumen are described.

### BACKGROUND

Stents are commonly used to maintain the patency of body lumens. For example, they are used in various arteries (e.g., coronary, peripheral, neck, and cerebral), in the veins, biliary ducts, urethras, ureters, fallopian tubes, bronchial tubes, tracheas, esophagi, and even prostrates. Stents are perhaps most often used in conjunction with angioplasty, to treat atherosclerosis, a cardiovascular disease characterized by the progressive narrowing and hardening of the arteries. Angioplasty, sometimes referred to as percutaneous coronary intervention (PCI), or percutaneous transluminal coronary angioplasty (PTCA) is a procedure in which a balloon (often placed on the distal tip of a catheter) is used to push back some of the plaque, which has built up on the artery wall.

In general, implanted stents help maintain the integrity of a lumen and prevent it from narrowing or closure. Because stents have provided continued lumen patency, and reduced rates of repeat revascularization, a number of different stent designs have emerged. Indeed, there are over 30 different stent designs currently in commercial use. These designs are often classified by their repeating pattern of metal construction (i.e., slotted tube, coil, or mesh), or by the nature of their delivery (i.e., self-expandable or balloon-expandable). Exemplary commercial stents include the Palmaz-Schatz Crown stent, the NIR stent, the MultiLink Duet, the In-Flow GoldFlex stent, the Bestent, the Terumo stent, the Crossflex LC, the GFX stent, the Wallstent, and the Jostents (for bifurcated lesions).

A recurring problem with stenting in vascular sites is restenosis, or the re-narrowing of the stented artery lumen after stent implantation. About 40 percent of all patients having stent implantation suffer some degree of restenosis within six months of the implantation. Some believe restenosis to be caused by new vessel wall tissue growth triggered by injury occurring as a result of the angioplasty or stent implantation procedures. Vessels suffering from restenosis often require subsequent angioplasty or surgery.

To combat restenosis, stents eluting anti-proliferative or anti-inflammatory drugs have been developed. These stents appear to have reduced the rate of restenosis, however the long term effects of the eluted drugs on the body have not yet been determined. In addition, these drug eluting stents were developed for use with traditional stents, such as those mentioned above, many of which, due to their size, shape, and method of deployment, remain undesirable for implantation in particular body lumens. Indeed, some of the above mentioned stents are of such a size, or are of such rigidity, that maneuvering them through tortuous vessels, or vessels having small circumferences is not only problematic, but virtually impossible. These issues are compounded when the vessels are in locations that are hard to access (e.g., intracranial vessels).

Indeed, a major problem faced during stent implantation is maneuvering of the stent through the body's access passageways to a target site (e.g. a lesion) without causing injury. Because stents are usually carried to a target site using catheters and guidewires, the profile of the delivery assembly may approach the diameter of the stent itself. The stent, folded or compressed to allow access through these passageways, is quite stiff. In addition to what might be considered the normal level of care in minimizing injury during such delivery, in the case of atherosclerotic vessels, special care must be taken to minimize the possibility of dislodging plaque, which could potentially result in the formation of an embolism and hence a “vascular accident.”

Also, the delivery and deployment of one or more stents often requires the changing of guidewires and the insertion of additional stents during the procedure. This in turn requires additional removal and replacement of the various devices, which raises the potential for infection. In addition, many of the known stents listed above are not amenable, with ease in any case, to placement against a lumen wall having a varying profile.

Avoidance of multiple removal and replacement steps as a means of preventing infection is desirable. Similarly, stenting structures having narrow profiles and increased flexibility would be desirable. In addition, stenting structures capable of readily contacting the walls of a lumen having a varying profile would also be desirable.

### SUMMARY

Described here are devices for delivering multiple prostheses having stenting properties within a body lumen. More specifically, multiple stent precursor assemblies comprising more than one elongated stent precursor member are described. In some instances, the stent precursor members are positioned at least partially side-by-side to the others to form a bundle, each stent precursor member configured to form stenting structures in situ at a site in a body lumen, and releasably adherent to the bundle. In some variations, the stent precursor members are wire like. The stent precursor members may be configured to release simultaneously from the bundle, and may be configured to release sequentially from the bundle. The multiple stent precursor assembly may comprise a guide member. The multiple stent precursor assembly may also comprise one or more clasps (e.g., an electrolytic clasp) releasably holding the stent precursor members so that they are not released from the bundle. The stent precursor members may be configured to release a sequence of simultaneously released stent precursor members from the bundle.

In some variations, the multiple stent precursor assembly comprises at least one stent precursor member that is self-forming into the stent structure. The self-forming stent precursor member may comprises a super-elastic material, such as nickel-titanium alloys, copper-zinc alloys, and nickel-aluminum alloys. In some instances, the super-elastic material comprises nitinol. In other variations, the multiple stent precursor assembly comprises at least one stent precursor member that is self-expanding into the stent structure. The self-expanding stent precursor member may comprise a super-elastic material, such as nickel-titanium alloys, copper-zinc alloys, and nickel-aluminum alloys.

In other variations, the at least one stent precursor member comprises a plastic material and is of dimensions such that the at least one stent precursor member is plastically deformed upon forming the stent structure. The plastic material may be those such as stainless steels, polyurethanes, ethers, acrylates, olefins, propylene, butenes, butadiene, styrene, and thermoplastic olefin elastomers, polydimethyl siloxane-based polymers, polyethyleneterephthalate, cross-linked polymers, non-cross linked polymers, rayon, cellulose, cellulose derivatives, nitrocellulose, natural rubbers, polyesters, lactides, glycolides, caprolactones and their copolymers and acid derivatives, hydroxybutyrate and polyhydroxyvalerate and their copolymers, polyether esters, anhydrides, hexadecandioic acid, and orthoesters. In some variations, the plastic material comprises stainless steel. In other variations, the plastic material comprises a polymer. Similarly, in some variations described herein, the at least one stent precursor member includes at least a first portion comprising a super-elastic material and at least a second portion comprising a plastic material and having dimensions such that the at least the second portion is plastically deformed upon forming the stent structure.

In still other variations, the at least one stent precursor member contains a drug, for example, anti-proliferation agents, anti-inflammatory agents, antibiotics, or immunosuppressants. Specific drugs suitable for use with the described devices include paclitaxel, methotrexate, batimastal, doxycycline, tetracycline, rapamycin, actinomycin, dexamethosone, methyl prednisolone, nitroprussides, estrogen, and estradiols.

In other variations, the multiple stent precursor assembly comprises at least one guide member and more than one elongated stent precursor members that are situated distal end to proximal end and are releasdeably adherent to the guide member. Each stent precursor member may be configured to be formable into a stent structure in the body lumen by a longitudinal movement with respect to a cooperating delivery element. In some variations, each stent precursor member is wire-like. In other variations, the stent precursor members is adherent to the guide member substantially along the length of that at least one stent precursor member.

The stent precursor members may be configured to release sequentially from the guide member. The multiple stent precursor assembly may comprise one or more clasps that releasably hold the stent precursor members to the bundle.

### BRIEF DESCRIPTION OF DRAWINGS

FIG. 1A is an illustration of an exemplary delivery device useful for delivering the stent precursor members described herein.

FIG. 1B provides an exploded view of an illustrative stent precursor structure described herein.

FIGS. 2A-21 provide illustrative cross sections of stent precursor member configurations.

FIGS. 3A-3C provide various side and cross-sectional views of some illustrative stent precursor members described herein.

FIGS. 4A-4E provide illustrative views of various stent precursor members detachably coupled to guide members.

FIGS. 5A-5E provide side and cross-sectional views of various shape forming members described herein.

FIGS. 6A and 6B show an illustrative stent precursor structure having multiple stent precursor members.

FIGS. 7A-7D show one variation in which a clasp may be used to hold the multiple stent precursor members in place prior to deployment.

FIGS. 8A-8B depict a typical electrolytic joint and its operation in a clasp to selectively release multiple stent precursor members.

FIGS. 9A-9D illustrate the use of multiple stent precursor members with a single guide member to form multiple stenting structures during a single procedure.

FIG. 9E provides a longitudinal sectional view of FIG. 9D.

FIGS. 10A-10B show various configurations of stent precursor structures having multiple stent precursor members.

FIG. 11 depicts a bundle of stent precursor members and its use as a guidewire.

FIGS. 12A-12D show various configurations in which the stent precursor member may be extended distally from a delivery device while the guide member is returned proximally.

FIG. 13 shows one variation of the described device with a balloon catheter.

FIGS. 14A-14E illustrate a typical method of using the described device with a balloon catheter.

FIGS. 15A-15D illustrate a method in which a balloon catheter may be used to deliver and deploy a stent precursor member.

FIGS. 16A-16D provide an illustration of one method of delivering and deploying stent precursor members to form stenting structures as described herein.

FIG. 17 provides an illustration of a balloon device that may be used to secure the stenting structure described herein to a wall of a body lumen.

### DETAILED DESCRIPTION

Described herein are devices and methods for delivery, deployment, and formation in situ of one or more stent precursor members to form stenting structures within a body lumen at a target site. The stent precursor members may be delivered and deployed, as desired, within a number of body lumens and at many desirable target sites. For example, the stent precursor members may be delivered and deployed within a body lumen of the arterial system, such as a body lumen within the coronary arteries, the peripheral arteries, and the cerebral arteries. Similarly, the stent precursor members may be delivered and deployed in the prostate via the prostatic urethra, the fallopian tube via its lumen, and any other suitable body lumen. The stent precursor members may be delivered to more than one target site, and deployed within more than one body lumen.

The target site may be a site within the body lumen where it is desirable to form a stenting structure. For example, in the case of forming stenting structures within the vasculature, the target site may be a stenotic or other diseased region. The target site may also be one which has previously been treated, for example, by conventional angioplasty procedures, artherectomy, laser angioplasty, ultrasonic ablation, or even one that has been previously stented. The target site may also be one that is suspected of being diseased.

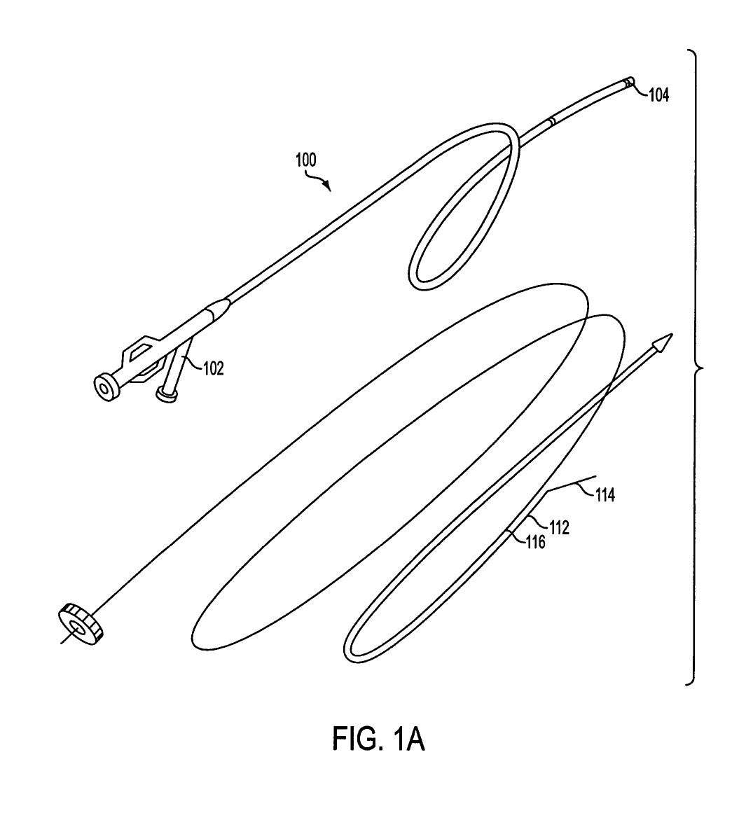

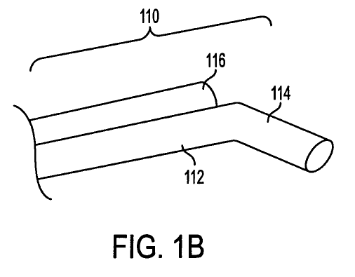

In general, our device includes a stent precursor structure for forming a structure in situ, at a target site within a body lumen where the resulting structure, referred to herein as a “stenting structure,” provides support to the lumen wall. Making reference now to the figures, wherein like numerals indicate like elements throughout the views, FIG. 1A, shows a delivery device or assembly (100) useful in the delivery of our stent precursor structures. To enhance understanding of our device and procedures, FIG. 1A shows one variation of a suitable delivery device (100) for delivering the stent precursor members in situ, here exemplified with a stent precursor member (114) and a guide member (116) to support the stent precursor member during delivery. The variation shown in FIG. 1A, depicts a delivery device (100), such as a catheter, a microcatheter, or other delivery device, having a Y-port (102) thereon. The combination of stent precursor member (114) and a guide member (116) are inserted into the delivery device (100). The choice of delivery device (e.g., size, etc.) is made depending on the nature and location of the target site to be treated. To permit location using fluoroscopy during a procedure, the delivery device (100) may include one or more radio-opaque bands (104) at or near its distal end.

The delivery device (100) may be inserted into an entry point in the patient's body at a location remote from the target site. For example, in the case where the target site is within the vasculature, one typical entry point is into the femoral artery of the groin. In essence, the delivery device (100) forms a passage way between the treating physician and the target site. Once the distal end of the delivery device (100) is positioned near the selected target site, the stent precursor structure (in this variation having a stent precursor member and a guide member) is inserted into the lumen of the delivery device and advanced therethrough. In this variation, the stent precursor structure is advanced past the distal end of the delivery device (100), and past the target site. This method of delivery is described in more detail below.

FIG. 1B provides an exploded view of the distal end of stent precursor structure (110). Stent precursor structure (110) comprises a delivery element (112) having a stent precursor member (114) releasably attached thereto. In some variations the delivery element (112) is non-caged. As we use the term here, the term “non-caged” means that the stent precursor member (114) maintains its shape by a support, or other member, which is not in the form of a tubular restraint, such as a catheter or a sheath. That is, the stent precursor member (114) is capable of being pushed distally past the target site in a deployable form, and may even contact the walls of the body lumen, without being deployed. The stent precursor structure (110) may optionally be secured to at least one guide member (116) along at least a longititudinal portion of the delivery element (112).

In general, the stent precursor member (114) becomes a coiled or helical stenting structure after detachment from the delivery element (112) or guide member (116), for example, by the methods discussed below. The stenting structure formed from the precursor member (114) comprises at least one turn, but the axial length, diameter, number of turns, and distance between adjacent turns can be controlled. Each turn need not have the same pitch or diameter as the previous one. The stenting structure may comprise any number of turns and comprise any number of shapes (e.g., circles, ovals, ellipses, etc), however shapes allowing the outer diameter of the stenting structure to easily conform to the wall of the body lumen may be more desirable. It is this flexibility of operation that permits some variations of this device to conform to a varying lumen wall diameter with such ease.

The stent precursor member (114) may be made of a variety of suitable materials and be of any of a wide number of configurations. Indeed, in certain instances, it may be desirable to have the stent precursor member constructed in such a way, and of such a material, that it is substantially self-forming. As used herein, the term “self-forming” means that the stenting structure formed when the stent precursor member (114) is detached from the delivery element (112) is of a predetermined configuration. This predetermined configuration for example, is determined prior to introducing the stent precursor structure into the delivery device (100). For instance, one example of a self-forming stent precursor member would be one comprising a superelastic alloy, or the like. Similarly, a “self-expanding” stent precursor member (a subset of “self-forming” members) may expand in diameter upon release without further action by the user. In this way, a helical or coiled stenting structure may be pre-formed prior to introducing the stent precursor structure into the delivery device. Similarly, the stent precursor member (114) may be made “semi self-forming.” That is, at least one section of the stent precursor member may comprise a material that is self-forming, while other sections of the stent precursor member are not.

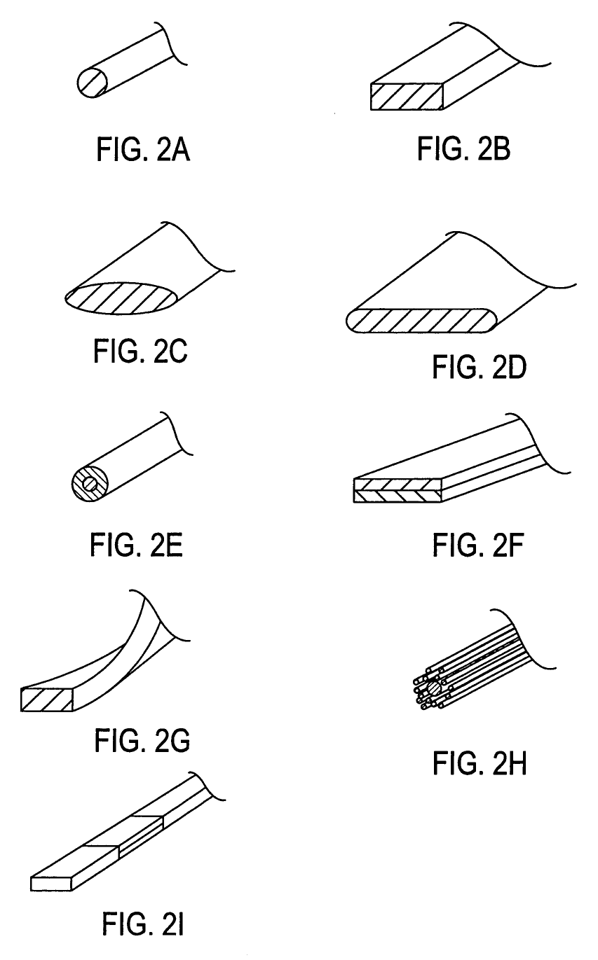

The stent precursor member (114) may also be “plastic” in nature. That is, the stent precursor member may be of such a size and be made of such a material, that when it is deployed to form the stenting structure at the target site within the body lumen, the forces associated with the deployment steps described herein below, will create plastic deformation in the stent precursor member (114). Obviously, the number and types of stent precursor members is only limited by the desired design and its subsequent utility. Any number of configurations, or combinations of configurations may be used. A few illustrative stent precursor member cross-sectional configurations are provided in FIGS. 2A-2I.

For example, the stent precursor member may have a rod-like or cylindrical figuration as shown in FIG. 2A, or it may have a rectangular configuration as shown in FIG. 2B. Similarly the stent precursor member may have an oval or elliptical type configuration as shown in FIG. 2C, or it may have a configuration having flattened top and bottom portions, with rounded sides, as shown in FIG. 2D.

FIG. 2E shows another variation of the stent precursor member having a first central portion surrounded by second outer portion, e.g., one or more coatings. This could, for example, allow for the use of multiple materials to impart various desirable properties. For instance, the stent precursor member may have a central portion, comprising a plastic or super-elastic metal or alloy, and then be coated with a composition containing a biologically active agent or the like. The coating may be a polymeric material having significant flexibility or, if desirable, the coating may comprise a harder material, providing less flexibility. While shown here in FIG. 2E as having a rod-like or coaxial cylindrical configuration, any number of shapes may be used, which allow for the use of multiple materials. For example, FIG. 2F shows one variation in which the stent precursor member has a first top portion, and a second bottom portion. As in the case with the coaxial configuration of FIG. 2E, the top and bottom portions may comprise different materials. Again, while FIG. 2F illustrates a stent precursor member having a first top portion and a second bottom portion in a rectangular configuration, any number of shapes may be used.

FIG. 2G shows yet another variation of the stent precursor member in which the stent precursor member is twisted. That is, it may have one or more bends or turns, giving it somewhat of a helical configuration. FIG. 2H shows a variation of the stent precursor member having a cable-like configuration. As shown in FIG. 2H, the stent precursor member can have a first inner portion, and a second outer portion comprising a multiplicity of smaller cylindrical configurations. This type of configuration may be advantageous for instance, to facilitate drug delivery. For example, each smaller cylindrical configuration may comprise a biocompatible polymer having a drug thereon. In this way, controlled and/or sustained drug delivery may be facilitated. Any number of biocompatible polymers and drugs may be used as described in more detail below.

FIG. 2I shows yet another variation in which the stent precursor member is made of more than one material, for example, longitudinally along its length. In this way, a combination of materials may be used to design a stent precursor member having a variety of desirable properties. For example, the materials may be a combination of alloys, a combination of super-elastic alloys, various plastics or polymers, or a mixture of any of the above. In addition, the dissimilar materials could be those having different coefficients of thermal expansion, forming, for example, a bimetal strip or section. In this way, the stent precursor member may be configured to form a given shape once the stent precursor member reaches a given temperature, or range of temperatures. Such sections may also comprise pre-formed sections of shape-memory metals or alloys that change shape upon entering the warmth of the human body.

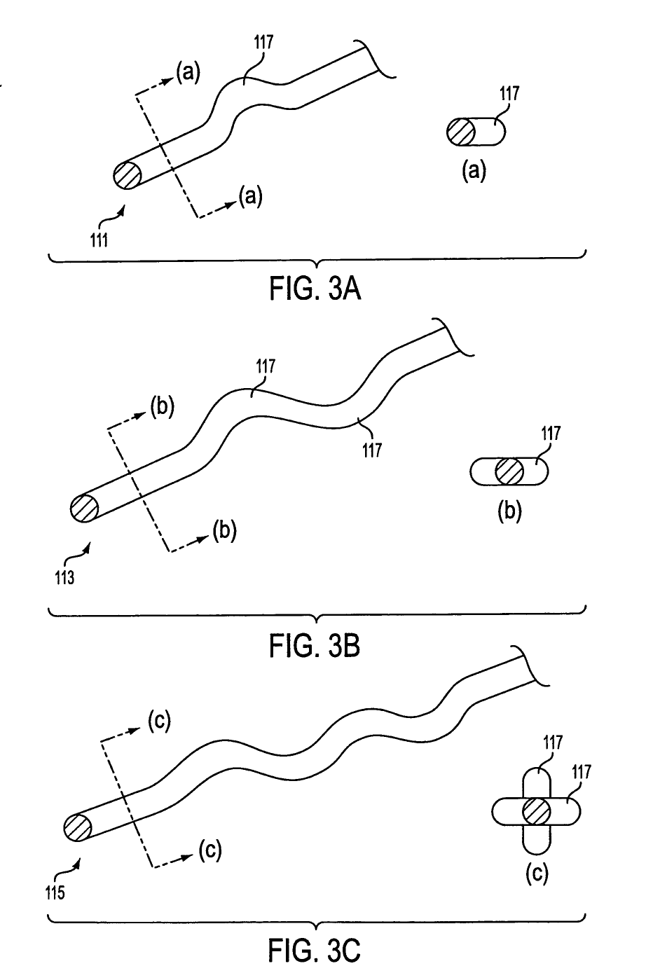

FIGS. 3A-3C show various side and cross-sectional views of exemplary stent precursor members having bends or undulations perhaps useful in anchoring the resulting stent structures at the desired body site. For example, as shown in FIG. 3A, the stent precursor member (111) is wire and has a bend (117) in a single plane, in a single direction. FIGS. 3B and 3C provide additional variations in which the stent precursor member is configured to have various other bending configurations. For example, a stent precursor member (113) having bends (117) in a single plane in two different directions is shown in FIG. 3B. A stent precursor member (115) having bends (117) in two planes and directions, generally orthogonal is shown in FIG. 3C. The bends need not necessarily be orthogonal to each other, of course. These types of configurations may be accomplished, for instance, by pre-bending a formable or plastic alloy, metal or other material or by the incorporation of more than one material into the stent precursor member. For example, a bi-metal section, a super-elastic alloy section, or combination of alloys may be used, which provide the deployed external configurations shown in FIGS. 3A, 3B, and 3C upon activation.

As noted above, the stent precursor member (114) may be made from a variety of materials, and need not be made of the same material as the delivery element (112) or optional guide member (116). For example, any biocompatible, non-toxic material imparting any of the desired properties discussed above, such as flexibility, etc., may be used. The stent precursor member (114) may me made of metals, metal alloys such as stainless steel, alloys having superelastic properties, polymers, or any of these in combination.

Examples of a suitable superelastic alloys include nickel titanium alloys (e.g., 48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Widely used NiTi alloys, generally known as “nitinol,” are those described in U.S. Pat. Nos. 3,174,851; 3,351,463; and 3,753,700, each of which is hereby incorporated by reference. Such an alloy tolerates significant flexinng even when drawn as a very small diameter wire. The formation of coil stents from nitinol alloys having both superelastic and shape memory properties is well known in the art, and described in U.S. Pat. Nos. 4,795,458 and 5,037,427, and PCT publication WO 94/16629, each of which is hereby incorporated by reference.

The stent precursor member (114), delivery element (112), and optional guide member (116) may also comprise or include a wide variety of synthetic and natural polymers, such as polyurethanes (including copolymers with soft segments containing esters, ethers and carbonates), ethers, acrylates (including cyanoacrylates), olefins (including polymers and copolymers of ethylene, propylene, butenes, butadiene, styrene, and thermoplastic olefin elastomers), polydimethyl siloxane-based polymers, polyethyleneterephthalate, cross-linked polymers, non-cross linked polymers, rayon, cellulose, cellulose derivatives such nitrocellulose, natural rubbers, polyesters such as lactides, glycolides, caprolactones and their copolymers and acid derivatives, hydroxybutyrate and polyhydroxyvalerate and their copolymers, polyether esters such as polydioxinone, anhydrides such as polymers and copolymers of sebacic acid, hexadecandioic acid and other diacids, orthoesters may be used. Activatable polymeric materials may also be included, for example thioisocyanates, aldehydes, isocyanates, divinyl compounds, epoxides or acrylates. Several of these polymers are biodegradable.

In addition to the aforementioned, photoactivatable crosslinkable groups, succinimidyl azido salicylate, succinimidyl-azidobenzoate, succinimidyl dithio acetate, azidoiodobenzene, fluoro nitrophenylazide, salicylate azides, benzophenone-maleimide, and the like may be used as photoactivatable crosslinking reagents. The activatable material may also consist of a thin coating which can be activated by external forces such as laser, radio-frequency, ultrasound or the like, with the same hardening result taking place. These materials would allow for normal tissue ingrowth to take place.

As noted above, the stent precursor member (114) may be coated, loaded, contacted with, or otherwise made to release a biologically active agent. For example, stent precursor member (114) may be coated with anti-proliferation agents, anti-inflammatory agents, antibiotics, immunosuppresants, as well as others, each of which may be used alone, or in combination with other active agents. Examples of suitable active agent include paclitaxel, methotrexate, batimastal, doxycycline, tetracycline, rapamycin, actinomycin, dexamethosone, methyl prednisolone, nitroprussides, estrogen, estradiols, and the like.

The stent precursor member (114) and delivery element (112) may also include one or more radio-opaque materials, in order to help facilitate delivery and deployment of the stent precursor member to form a stenting structure. Examples of suitable radio-opaque materials include platinum, rhodium, palladium, rhenium, as tungsten, gold, silver, tantalum, or any alloys of these metals. Selection of an appropriate radio-opaque material, or combination of radio-opaque materials, may be made based on the degree of flexibility or stiffness desired.

In general, when the stent precursor member (114) is made of a metallic material, such as a stainless steel alloy or a superelastic alloy such as nitinol, the diameter of the precursor member used to form the stenting structure may be in the range of 0.0001 and 0.05 inches. Similarly, when the stent precursor member is formed from a stainless steel or nitinol wire, which is to be additionally coated (e.g., with a radio-opaque material), the diameter of the wire may be in the range of 0.001 to 0.02 inches. The stenting structure may than have outer diameters ranging between 0.005 and 0.025 inches. In any event, the diameter of the stenting structure formed in situ should be of an appropriate and sufficient size so that the stenting structure is held in place within the chosen lumen typically without substantially, or inappropriately, distending the lumen walls. In cases where the stenting structure is to be placed within the vasculature, it should be of a sufficient diameter to withstand the repetitive pulsing of the vascular system. However, as noted above, the diameter of the stenting structure need not be uniform along its length, it may be variable.

The stent precursor member (114) may be made detachable from the delivery element (112) and/or optional guide member (116) by a number of different mechanisms. For example, the stent precursor member, delivery element, or guide member may include a sufficient amount of one or more electrically conductive materials at a desirable point of detachment. In this way an electrolytic joint is formed and the stent precursor member (114) is made detachable from the delivery element or guide member, and a pre-selected length of stenting structure is achieved. U.S. Pat. No. 5,354,295 and its parent, U.S. Pat. No. 5,122,136 (and a large number of other patents) to Guglielmi et al, describe an electrolytic detachment mechanism, each of which is hereby incorporated by reference. The length of stent precursor member (114) may also be made sufficiently long so that it may be divided into a number of individual stenting structure, for example in the range of 2-5, or more. Other suitable mechanisms of detaching the stent precursor member include the use of ultrasound, radio-frequency, screw-type connections, hydraulic detachment mechanisms, heat-activated thermoplastic containig joints, and mechanical detachment mechanisms, for example, those described in U.S. Pat. No. 5,234,437, to Sepetka, U.S. Pat. Nos. 5,250,071 and 5,312,415, to Palermo, U.S. Pat. No. 5,261,916, to Engelson, and U.S. Pat. No. 5,304,195, to Twyford et al.

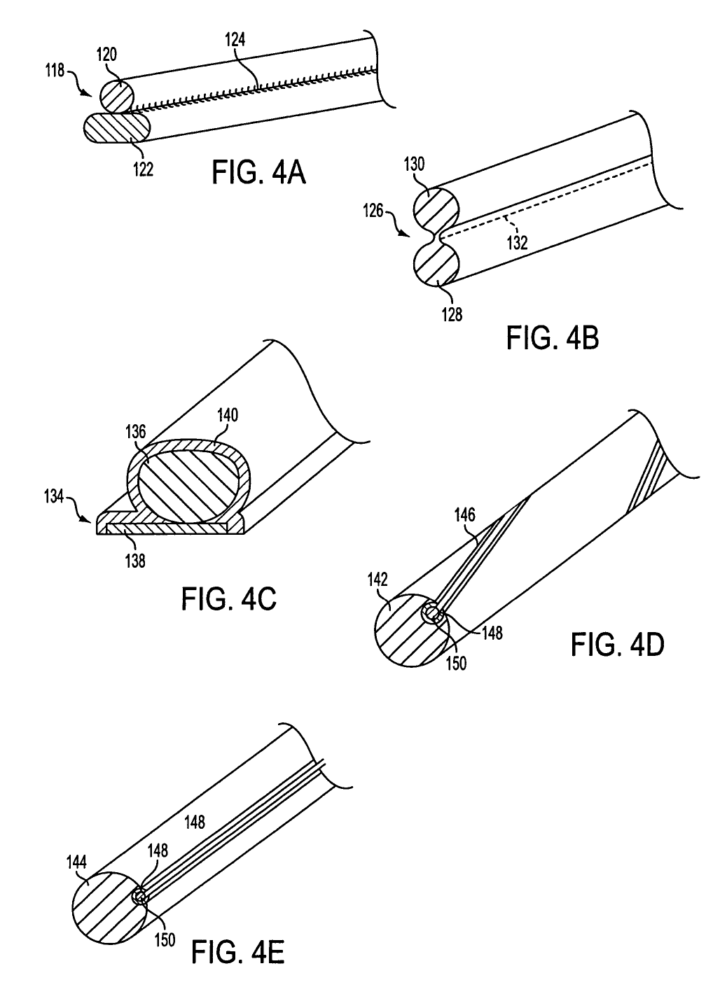

As noted above, a guide member (116) may be employed. In these variations, the stent precursor structure (110) may be made to adhere to, or to be conjoined with, the guide member (116). FIG. 4A shows a structure (118) comprising stent precursor member (120) and a guide member (122). In this variation, a seam of adhesive or glue (124) is shown causing adherence between the stent precursor member (120) and the guide member (122). Although the line of adhesive (124) is shown to be reasonably continuous, it may be either semi-continuous or at intervals along the length of the two members (120, 122).

As is the case with any of the variations discussed herein, some prior thought must be had in selecting the type of adhering material, the amount of adhering material, and the appropriate placement for the adhering material. For example, in some variations of the described device, the stent precursor member (120) is peeled away from its attendant guide member (120). This “peeling” may be done, for example, by using a catheter tip, perhaps with a forming member (as discussed in more detail below). Consequently, a reasonable designer would choose a type and amount of adhering material that, in addition to being biocompatible and non-toxic, would have a low peel strength in order to allow the stent precursor member (120) to easily detach from the guide member (122) and be delivered to the target site, without incident. Similarly, it is desirable that the adhering material be chosen with an eye towards maintaining the unseparated adhering material on the guide member (122). In this way, the guide member (122) will retain the adhering material thereon until later removal, so that the adhering material will not be released into the body lumen where the stenting structure is to be formed. This in turn minimizes the risk of creating embolisms. Chemical treatments to modify the adherence of glues and other binding agents to substrates are well known, and may be used to enhance or lessen the adherence of the adhering material to the stent precursor member (120) or guide member (122).

FIG. 4B shows another combination (126) of a guide member (128) and stent precursor member (130). In this variation, the two elements (128, 130) are not joined together by an additional material, but, are instead, pieces that are separable from a single element having a separation or “tear” line (132) between the two sections. Obviously, such a frangible joint is easier to provide for using readily moldable materials, such as thermoplastics. However, such a section may be formed by rolling and punching a metallic pre-form (e.g., a wire or ribbon), despite the normally modest size of the stent precursor structure to be formed.

FIG. 4C shows still another variation (134) in which the stent precursor member (136) is functionally attached to the guide member (138) using a covering (140) of material designed to be left at the target site. For instance, in some variations of the device, the guide member (138) transporting and translating the stent precursor member is returned into the delivery device or delivery system once it releases the stent precursor member. These variations are described in more detail below. However, the arrangement shown in FIG. 4C is suitable for such variations, wherein the guide member (138) would typically be comparatively fairly flexible. Similarly, the covering (140) may be used to carry anti-restenosis drugs or the like or may be produced from biocompatible and body-fluid-soluble glue such as one comprising fibrin. Such materials may provide a pharmaceutical benefit, may help maintain the position of the resulting stenting structure within the body, or may simply be dissolved over time without causing particular harm.

FIGS. 4D and 4E show additional variations of the described guide-stent precursor member, which use either modest mechanical clamping action by the guide members respectively (142 and 144), or which utilize small amounts of an adhesive or positioning material (146) in slots or grooves (148) provided along the exterior surface of the guide member (142, 144). The slots or grooves (148) are configured to allow placement of the stent precursor member (150) in each of the slots. FIG. 4D shows a variation in which the slot is cut in a helical fashion around the outer edge of guide member (142). It should be apparent that this helical slot (148) aids in the delivery of the stent precursor member (150) as it forms the stenting structure at the target site within a body lumen. That is to say, the placement of the precursor stent member along the inner circumference of the body lumen into which the stenting structure is to be formed may be more easily accomplished.

A shape forming member may be used to provide curvature to the stent precursor member as it is being delivered. As described in more detailed below, as the stent precursor member is pulled across the shape forming member, the precursor bends, providing a curve or turn in the stent precursor member. In this way, the stent precursor member is formed into a shaped, e.g., coiled or helical, stenting structure. The shape of the shape forming member is designed or selected depending on the desired shape of the stenting structure to be formed. For example, the shape forming member may be a wedge type of structure positioned on the outer surface of a delivery device, at or near its distal end. The wedge type structure may have at least one slanted surface and at least one horizontal surface joined thereto. The joinder of the two surfaces defines an angle, the degree of which may be selected so as to impart a desired final diameter to the stenting structure to be formed.

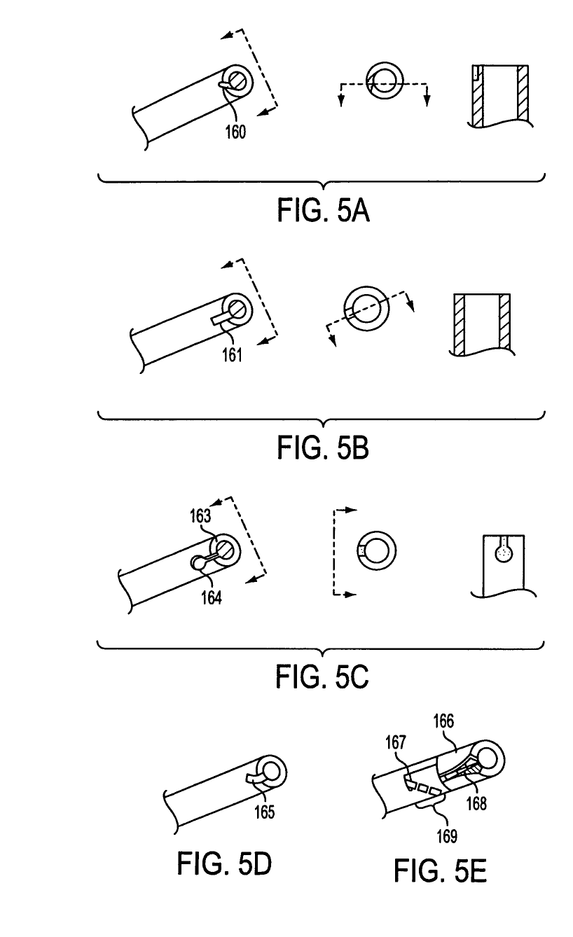

Other suitable variations of the shape forming member are shown in FIGS. 5A-E. In each of the variations depicted there, the shape forming member is positioned at the distal end of a delivery device, such as a catheter. Side, cross-sectional and top views are shown. The shape forming members depicted in these variations may be formed, for example, by providing a shaped slot of sorts, which extends from the lumen of the catheter to its outer surface as shown in the cross-sectional views. As the stent precursor member is pulled proximally past the shape forming member, the shape forming member imparts at least one curve or bend to the stent precursor member in a given direction. Although shown as structures that impart shape to the stent structure upon stent precursor movement that is proximal with respect to the shape forming member, the shape forming member may be reversed to impart shape when the stent precursor is moved distally to the shape forming member.

For example, as shown in FIG. 5A, the shape forming member (160) may be configured to provide a radial or tangential bend or direction to the stent precursor member pulled thereover. Similarly, the shape forming member (161) may be configured to provide a proximal bend or direction to the stent precursor member as depicted in FIG. 5B. FIG. 5C provides another variation in which the shape forming member (163) has a keyhole-like configuration, having an initial slot that extends into a wider structure, in this variation, shown as a circle (164). The circular structure (164) acts to capture the stent precursor member, and may therefore be designed to have dimensions that provide the final shape of the stenting structure desired to be formed. FIG. 5D shows another variation, similar to that of FIG. 5A, in which the shape forming member (165) provides a more radial or curved direction to the stent precursor member.

FIG. 5E shows yet another variation of the shape forming member, in which the angle imparted to the stent precursor member by the shape forming member may be made variable in situ during a stent forming procedure. As shown in FIG. 5E the shape forming member on the catheter has two portions, a distal portion (166) and a proximal portion (167). The proximal portion of the shape forming member (167) is positioned on the distal portion (166) and, in this depicted variation, includes a funnel-like area that intercepts the stent precursor and directs it to a forming or contact surface on the proximal portion. Positioned along the proximal portion (167) is a contact surface, here shown as a series of ramps (169). In this variation, each ramp is shown to have a different angle such that when the stent precursor member contacts the ramp surface, a given angle of deflection (and hence, forming) is given to the stent precursor transforming it into a stent structure. The proximal portion (176) of the catheter may be rotated or turned with respect to the distal section (166) as desired to facilitate contact of the stent precursor member with the desired ramp surface (169). In this way, the angle of the stent precursor member deflection may be adjusted in situ during the procedure. This permits adjustment of the size, pitch, etc. of the resultant stent structure without removal of the forming device from the body.

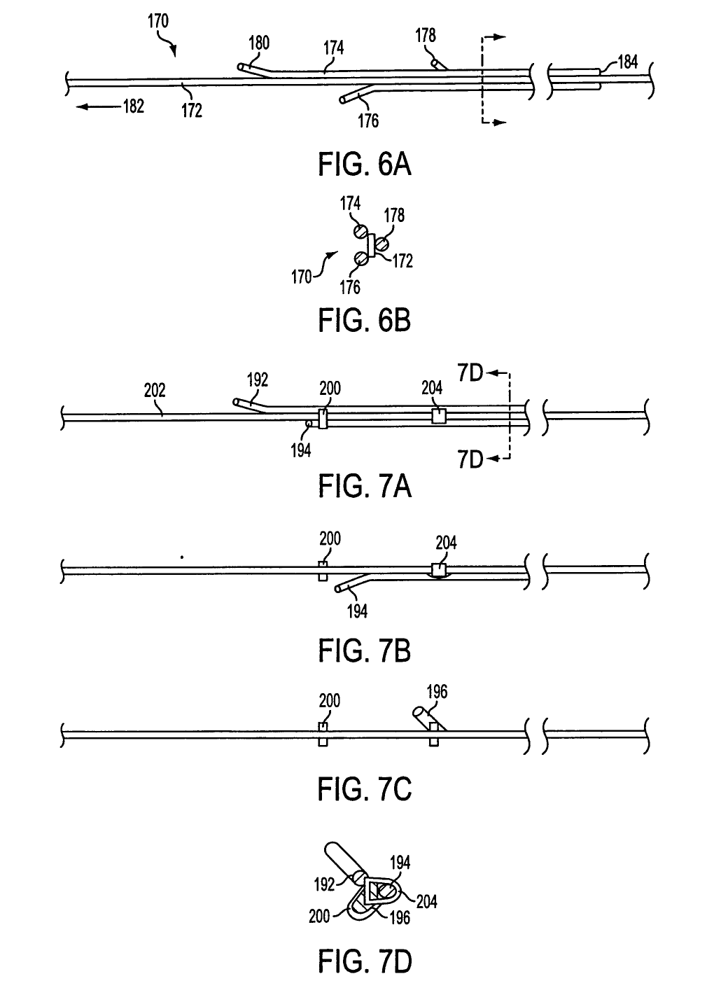

One advantage of this variation of the described device is that multiple stenting structures may be formed in situ simultaneously or sequentially, without removing the stent precursor structure from the delivery system. The device may form multiple stenting structures using any of the above described stent precursor structures, including those utilizing a guide member. For example, one device suitable for forming multiple stenting structures is depicted in FIGS. 6A and 6B.

The device (170) shown in FIG. 6A comprises a guide member (172) and a number of stent precursor members (174, 176, 178). In this variation, the device (170) is designed to allow the simultaneous deployment of multiple precursor stent members (174, 176, 178) during a single deployment movement. That is to say, that multiple stent precursor structures are placed in the deployment device and then advanced distally within the selected body lumen. Those stent precursors are substantially parallel for at least a portion of their overall length. These stent precursors may be considered in some variations to be “wire-like.” By “wire-like,” we mean that the in the central regions of the precursors, where the stent precursors are in general contact, the largest effective diameter (measurement across the broadest dimension of the stent precursor cross-section, e.g., as shown in FIGS. 2A to 2H) is less than about 0.200 inches, preferably less than about 0.100 inches. The stent precursor members to be deployed are then deployed as they are pulled proximally past the target site in the body lumen to be treated. For instance, if the surgeon using the device shown in FIG. 6A wished to deploy all three of the stent precursor members shown, the assembly (170) would be slid into the lumen so that the engagement region (180) of stent precursor member (174) was distal of the chosen target site. As guide member (172) is pulled proximally (182), the engagement region (180) of guide member (172) would first meet a shape forming member (not shown in this drawing). Additional proximal movement of guide member (172) would then cause stent precursor member (176) then to engage a shape forming member and begin to deploy; additional proximal movement (182) by guide member (172) would finally cause stent precursor member (178) to contact the shape forming member and begin the formation of a stenting structure made of three stent precursor members all deployed at substantially the same time. The shape forming members of this variation need not be one in the same. That is, each stent precursor member could have a dedicated shape forming member configured to contact the engagement region of its corresponding stent precursor member.

In essence, the assemblage (170) shown in FIG. 6A, is simply a multiplicity of a stent precursor members adherent to a guide member (172). The distal ends of the multiple stent precursor members may be positioned to terminate at the same place (184) as is shown in FIG. 6A, or the distal ends of those stent precursor members may be staggered or, at least, not terminate at the same location on the guide member (172). FIG. 6B is a cross-sectional view of the assemblage shown in FIG. 6A.

FIG. 7A shows an assembly (190) of multiple stent precursor members (192, 194, and 196) (192 is not shown in FIG. 7A, but visible in FIG. 7C and FIG. 7D). A major difference between the assembly (190) shown in FIGS. 7A-7D and that of (170) shown in FIGS. 6A-6B, is the use of clasps to hold one or more or all of the engagement regions associated with each stent precursor member away from any device that could begin a deployment of the stent precursor member, until such time as deployment is desired.

In concept, the variation of the assembly shown in FIG. 7A has a number of bands, each holding one less stent precursor member than the clasp or band proximal to it. This allows the deployment of the outer most stent precursor member, reintroduction of the partially depleted assembly (190) to allow deployment of a second stent precursor member (194) after its associated clasp or band (200) has been opened, and finally deployment of the final stent precursor member (192) after its clasp or band (204) has been opened. This sequence of events is shown in FIGS. 7A, 7B, and 7C, respectively.

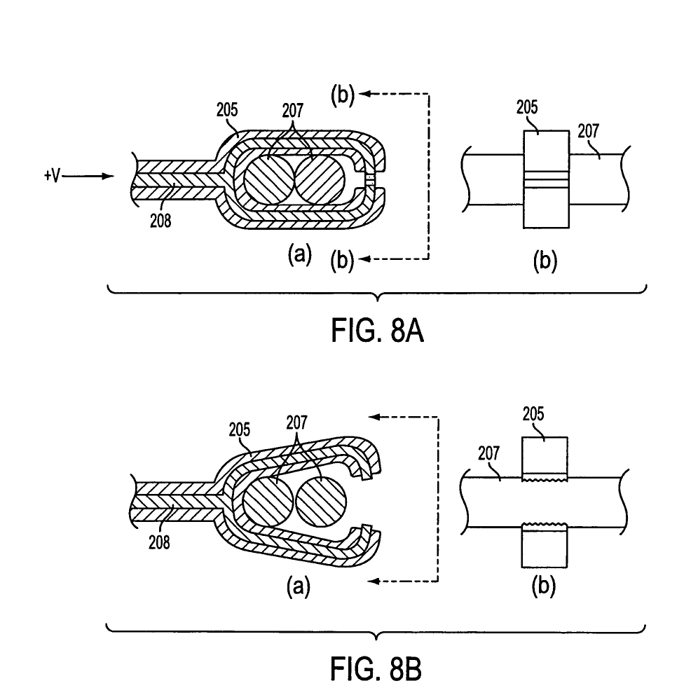

This device allows for the placement of a multileveled or multilayered stenting structure, a series of overlapping stenting structures, or a series of linearly placed stenting structures, all as desired without removing the deployment device from the patient's body. FIG. 7D shows a cross-section of the initially introduced device as found in FIG. 7A. Shown in FIG. 7D, are the guide member (202), clasp (204), clasp (200), and the three stent precursor members (192, 194, 196). Severing or opening clasps (200 and 204) may be readily performed using separate electrolytic joints, such as those described in detail below and shown in FIGS. 8A and 8B.

For example, FIGS. 8A and 8B show how an electrolytic joint may be used in conjunction with the above described clasps. As shown in FIG. 8A, the clasp (205) has an electrolytic joint (206). An electrically conductive wire (208) is used to transfer electricity from an external power supply (+V) to the electrolytic joint. The wire (208) may be constructed of a material so that it is insulated from the electrolytic joint itself. When the electrolytic joint has not been activated, the clasp is closed, holding the stent precursor members (207) securely in place, as shown from the side in FIG. 8A(a), and from the front in FIG. 8A(b). However, as illustrated in FIG. 8B, once energy is delivered to the joint, it dissolves, opening clasp (205) and allowing the stent precursor members (207) to be released. These types of joints are well known in the art, as described above and in the patents which were there incorporated by reference in their entirety.

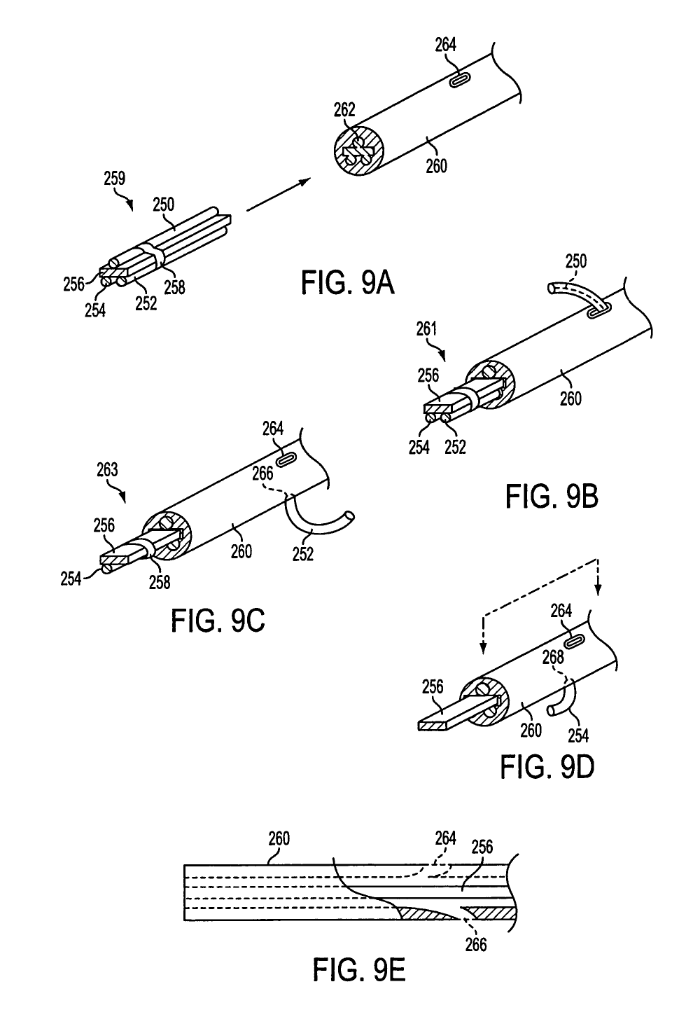

FIGS. 9A-9D illustrate how multiple stent precursor members may be used with a single guide member to form multiple stenting structures during a single procedure, using for example, electrolytic clasps of the type described above. For instance, in the variation shown FIG. 9A, stent precursor members (250, 252, and 254) are releasably attached to guide member (256) using for example, one or more electrolytic clasps (258) as described above. The assembly (259) of multiple stent precursor members (250, 252, 254) and guide member (256) is then advanced distally into the lumen (262) of a delivery device (260). In the variation shown in FIG. 9A, the lumen (262) is configured to accept three corresponding stent precursor members, however, the lumen may be designed so as to accept more or less, for example, from 2-5 stent precursor members.

The delivery device (260) has one or more ports (264) or openings thereon for receiving at least one stent precursor member therethrough. As shown in FIG. 9B, as the assembly (259) is advanced distally through the lumen (262) of delivery device (260), the stent precursor member (250) exits port (264). The assembly (259) is then withdrawn proximally and the electrolytic clasp (258) is dissolved, releasing stent precursor member (250) to form a stenting structure in situ. The guide member (256) in this variation acts as an indexing medium between the delivery device (260) and the assembly (259).

The assembly (261), having one less stent precursor member, may then be advanced distally, to delivery and deploy a second stent precursor member (252). Stent precursor member (252) is advanced distally where it exits port (266). As the assembly is withdrawn proximally, the stent precursor member (252) is deployed and begins to form a stenting structure. The electrolytic clasp is then dissolved.

In a similar fashion, the assembly (263), having only one remaining stent precursor member (254) may be advanced distally to deliver and deploy stent precursor member (254). As the assembly is withdrawn proximally, stent precursor member (254) is deployed. The last electrolytic clasp is then dissolved, releasing stent precursor member (254) and allowing it to form a stenting structure in situ. While the variations shown in FIGS. 9A-9D illustrate three different ports (264, 266, 268) for receiving three different stent precursor members (250, 252, and 254 respectively), this need not be so. For example, a single port may be used to receive each of the stent precursor members employed. FIG. 9E provides a longitudinal sectional view of FIG. 9D.

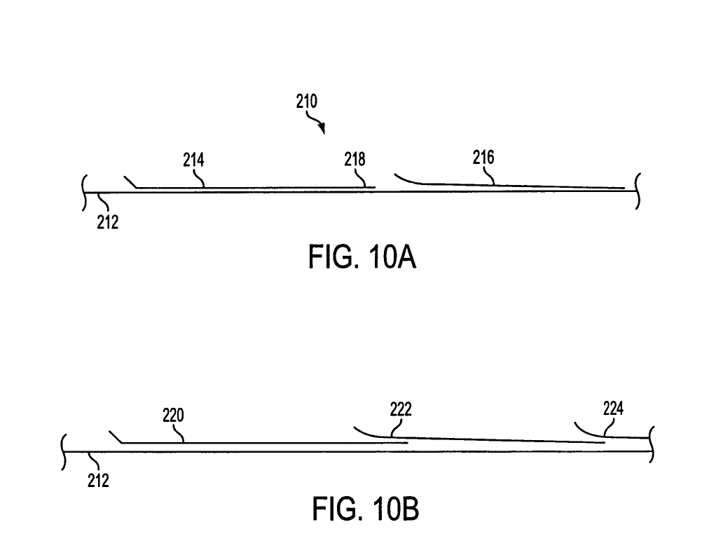

FIGS. 10A and 10B show additional variations of the described device configured to release multiple stent precursor members. FIG. 10A shows an assemblage (210) having a guide member (212) and a first stent precursor member (214). Second stent precursor member (216) is shown to be separated from the distal end (218) of the first stent precursor member (214). This configuration allows the user to fully deploy stent precursor member (214) completely before beginning deployment of stent precursor member (216).

In the generic representations shown in FIGS. 6A and 10A, the stent precursors may be of the normal columnar configurations, e.g., tubular in form and constructed of wire, tubes, or rolled and welded, and delivered as shown. We refer to those stent configurations specifically as tubular stent precursors.

FIG. 10B similarly shows in schematic fashion, a guide member (212), a first stent precursor member (220), a second stent precursor member (222), and an additional stent precursor member (224). In this configuration, the next-trailing stent overlaps the proximal end of the leading stent by a certain distance, to allow the user to begin deploying the following stent after the more leading stent is finished with its deployment.

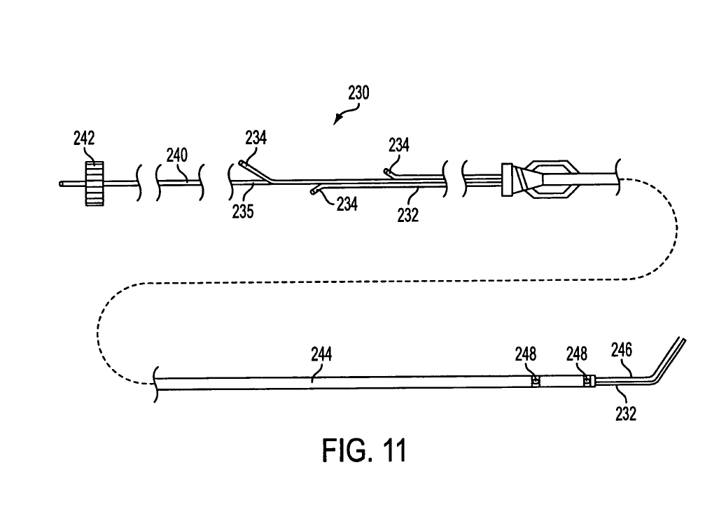

FIG. 11 shows an assembly (230) in which a bundle (232) of stent precursor members (234) are bound together, in a way similar to those discussed elsewhere here, which may achieve the function of a guide wire. In this variation, a guide member (235) is shown to support the bundle (232) of stent precursor members. This variation allows access of the device into areas having modest, perhaps minimal clearance, as might be found in the neural vasculature.

The structure (230) shown in FIG. 11 includes the bundle (232) of multiple stent precursor members (234) bound together as discussed above. Additionally, a sleeve (240) with an attendant handle (242) is shown. The sleeve (240) may slide over the proximal end of the assembly (232) of multiple stent precursor members (234). Catheter (244) designed to approach a lesion or other target site within a body lumen is shown with the distal portion (246) of bundle (232) emanating from its distal end. A pair of radio-opaque bands (248) may be placed at or near the distal end of catheter (244) to allow a user to determine where the distal end may be during a selected procedure, via fluoroscopy.

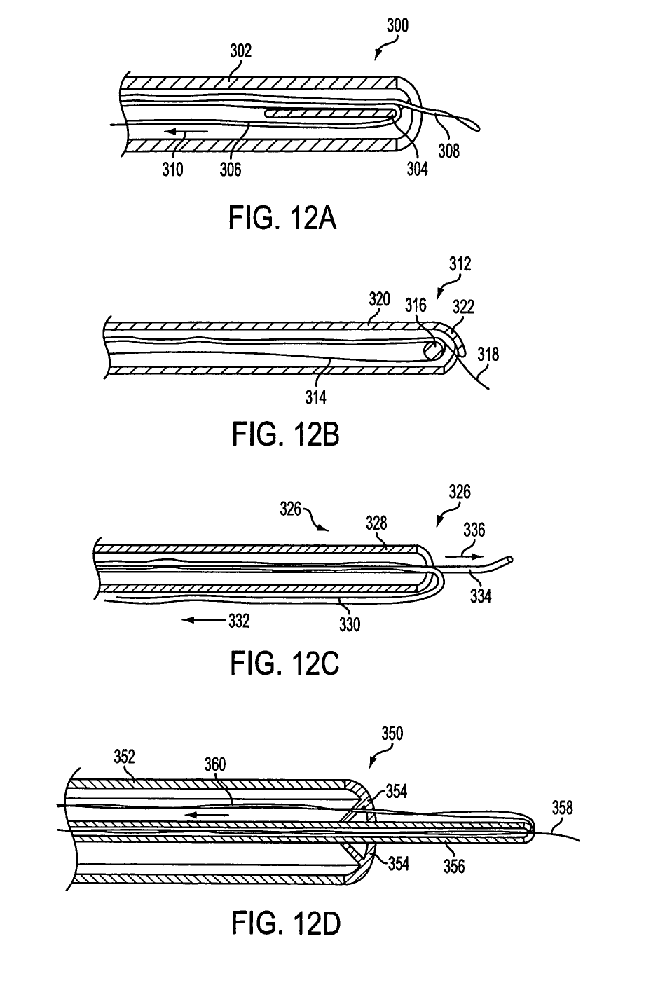

FIGS. 12A-12D, show a variation of the described device in which the stent precursor member is extended distally from a delivery device of some kind, while the support or guide member is returned proximally from the point where (or, at least near) the stent precursor member is separated from the guide member. This arrangement has certain benefits, the major one of which may be, that the stent precursor structure or any of its components, need not be advanced significantly past the target site. In a number of variations described herein, the delivery element or the guide member may pass the target site for a significant distance before deployment of the stent precursor member at that site.

FIG. 12A shows one variation of the disclosed device (300) in which the delivery member (302) is shown to be a catheter, or the like, having a separator wall (304) near its distal end. Separator wall (304) allows the guide member (306) to separate from the stent precursor member (308) and to pass back to the proximal end of the delivery device. The guide member or delivery element (306) is pulled in a proximal direction (310) allowing a separation or a release of the precursor stent element (308).

FIG. 12B shows a similar assembly (312) in which the guide member (314) passes around a circular turning post (316) to provide separation between the guide member (314) and the stent precursor member (318). The turning post (316) may be of any convenient shape allowing such a separation and lowering the overall friction of the turning operation. If convenient, the turning post (316) may rotate. The distal end of the delivery member (320) is shown to have a directing member (322) located distally, to direct the stent precursor member (318) to the target site. The directing member (322) may be of any convenient size or direction allowing or enhancing movement of the stent precursor member towards the wall of the target site.

FIG. 12C shows another assembly (326) in which the delivery device comprises a catheter body (328). In this variation, the guide member (330) is returned to the operator without the tubular member. Again, pulling the guide member (330) in a proximal direction (332) permits the stent precursor member (334) to exit the delivery device in a proximal direction (336).

It should be noted that the guide members in the variations discussed in regard to FIGS. 12A-12D may be significantly more flexible than other of the guide members discussed herein. For instance, in some variations of the described device, the guide member provides significant independent support to the stent precursor member. The support member in this variation desirably has significant shear strength when pulled from one end to the other, but may not have, for example, significant inherent stiffness.

A structure related in some concepts to those shown in FIGS. 12A-12C is shown in FIG. 12D. FIG. 12D shows a structure (350) that is comparably stiff in some ways to a typical cardiovascular or neurovascular guide or catheter. The proximal portion (352) of the assembly (350) desirably has internal walls (354) supporting an interior tubular portion (356) through which the stent precursor member (358) and the attached guide member pass. The variation shown in FIG. 12D includes an extension (356) that extends through the central passageway. Such a structure, with its low diameter nose piece (356) extending distally from the larger and stiffer main body section (352), may provide several advantages. The low diameter extension (356) may be extended into, and even distally of, a lesion or target site and may provide stiffness when the guide member (360) is pulled proximally to detach the stent precursor member.

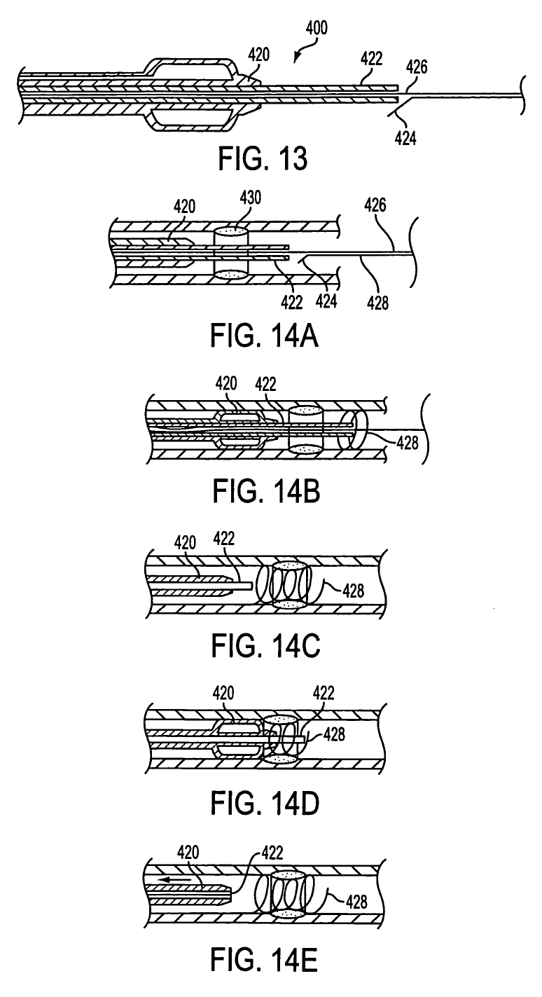

FIG. 13 shows a device (400) made up of a balloon catheter (420) and a tubular member (422) that is able to slide and to rotate within the inner bore of balloon catheter (420). The stent precursor member (424) and the guide member (426) are also shown. This variation may have a number of advantages that could be quite useful depending upon the circumstances of use. For instance, if the diameter of the lumen selected for treatment is quite variable, this variation includes a small balloon catheter that may be used to tailor or to size the stenting structure deployed at the target site. The inner tubular area (422), in addition to being used as a delivery element for separating the stent precursor member from the guide member, may also be used, in the manner of a guide “wire” and the deflated balloon catheter (420) may follow it into the region where the stenting structure was formed. The balloon may then be used to tailor the diameter of the resulting stenting structure, if desired.

Specifically, FIG. 14A depicts a lumen varying body organ such as an artery. A lesion (430) is shown interior to the artery. The stent precursor member (428) is shown to be introduced distally past lesion (430). Attached guide member (426) is also shown. The inner tubular catheter member (422) that serves, in this instance, as the delivery element has been extended distally past the target site (430) as well. Deflated balloon catheter (420) is shown proximal of the target site (430). In this variation, the balloon catheter (420) may be inflated to provide a specific amount of proximal/distal immobility to the resulting device. The tubular member (422) is drawn proximally, towards the operator, and the guide member (426) is similarly drawn proximally so that the stent precursor member (428) is cleaved from the guide member (426) and forms a stenting structure within the artery lumen. As shown in FIG. 14C, the guide member (426) and the inner tubular member (422) are withdrawn proximally as well. Since support is no longer needed, the balloon catheter (420) is deflated. Stent precursor member (428) has become a stenting structure.

In the event that additional shaping or forming of the resulting stenting structure (428) is desirable, the inner tubular member may be used as a guide (as shown in FIG. 14D) for balloon catheter (420). The balloon catheter (420) is inflated to reform the shape of the stenting structure. FIG. 14E shows the modified stenting structure (428). The balloon catheter (420) and the tubular member (422) are then withdrawn from the target site. This variation provides significant operational flexibility in that the small tubular member may be introduced through the treatment area for aiding in the longitudinal placement of the stenting structure. The central tubular member may also be used to provide a good passageway and direction for the balloon “clean up,” should one be needed.

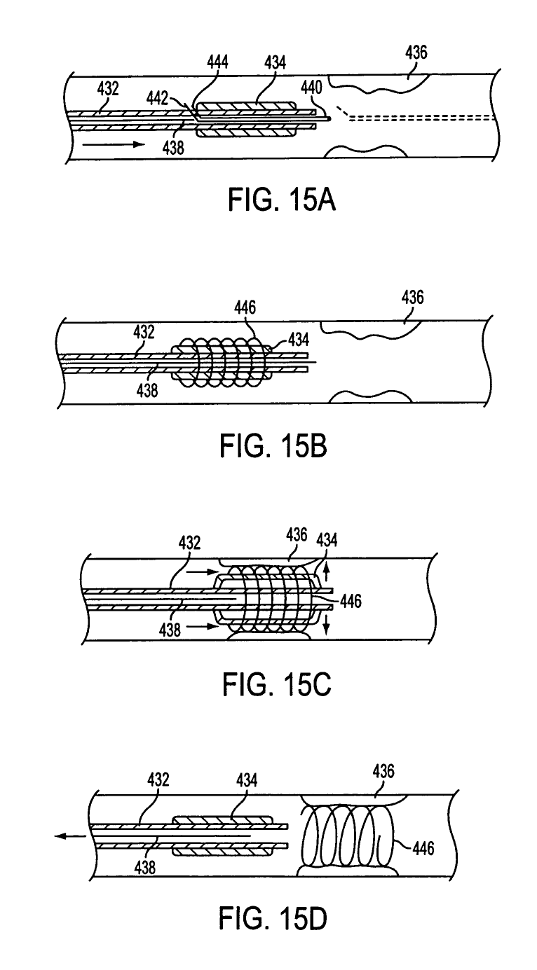

FIGS. 15A-D illustrate additional methods in which a balloon catheter may be used to deliver and deploy stent precursor members. For example, as shown in FIG. 15A, a delivery device (432) (e.g., a catheter) having an expandable balloon (434) thereon may be advanced distally through a body lumen toward a target site (436) for treatment. As shown in FIG. 15A, the balloon (434) is in a collapsed, deflated configuration. In the variation shown in FIG. 15A, a guide member (438) having a stent precursor member (440) releasably attached thereto is advanced distally through the lumen of delivery device (432). The stent precursor member (440) is advanced within the delivery device (432) until its engagement region (442) engages an opening or port within the delivery device, which allows it to exit the device near the proximal end of the deflated balloon (434).

As shown in FIG. 15B, the guide member (438) is pulled proximally causing the engagement region (442) to contact a shape forming member (444) positioned near the proximal end of the deflated balloon (434). As the guide member is pulled proximally, the stent precursor member (440) contacts the shape forming member (444) which provides curves or bends in the stent precursor member (440), thereby forming a helical or coiled structure that surrounds the collapsed balloon (434) as it is formed. The delivery device having the collapsed balloon with the surrounding coiled structure thereon, is then advanced distally toward the target site (436). Once the balloon is positioned across the target site (436), the balloon may then be expanded as shown in FIG. 15C. Appropriate positioning of the balloon may be accomplished by any of the fluoroscopy-aided techniques discussed elsewhere.

The inflation of the balloon (434) expands the lumen of the stenting structure (446) until its outer circumference gently contacts the walls of the target site. Suitable techniques for balloon inflation are well known in the art, and any such suitable techniques (e.g., use of pressured saline, etc.) may be used with the methods described here. After the stenting structure is anchored to the lumen walls of the target site, the balloon can be collapsed to its first non-expanded configuration as shown in FIG. 15D. The delivery device (432) may then be removed from the patient by withdrawing the device proximally.

Although FIG. 15A shows one variation in which the stent precursor member exits an opening or port of the delivery device, the stent precursor member need not begin forming a stenting structure in this fashion. For example, the stent precursor member may be advanced distally past the distal end of the delivery device (432) as shown by the dashed lines in FIG. 15A. In this variation, as the guide member (438) is pulled proximally, the engagement region of the stent precursor member contacts a shape forming member (not shown in this drawing) located perhaps at the distal-most portion of catheter (432) or between the distal end of the catheter and the balloon (434) and begins to form a stenting structure, in a proximal direction. As the guide member (438) is pulled proximally, the stenting structure continues to form around the deflated balloon (434). This procedure may be used to form stenting structures variously on the catheter shaft proximal of the balloon, distal of the balloon, on the balloon as inflated, on the balloon as deflated, on the balloon as partially inflated, or even in the open artery—in each case followed, as desired, with reforming of the stenting structure using the balloon.

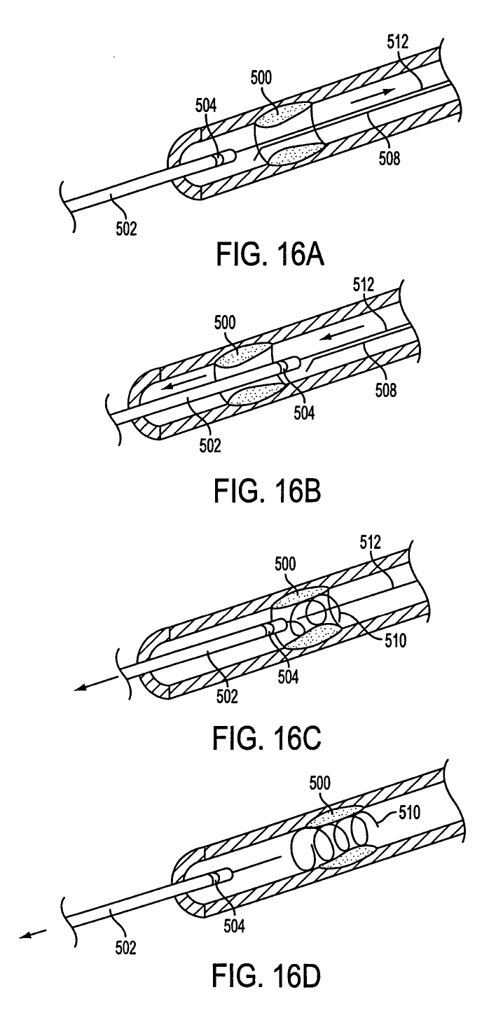

FIGS. 16A-D illustrate additional variations of delivering and deploying the stent precursor members described herein. For example, the target site (500) may be reached using a delivery device (502) having one or more radio-opaque markers (504) positioned on or near its distal end. As with any of the methods described herein, appropriate and specifically chosen catheters (e.g., microcatheters, neurovascular catheters, etc.) and guides may optionally be used to effectuate the procedure. The length and diameter of these optional catheters and guides is chosen based upon the location and size of the target site selected for treatment. For example, the catheter may have a length between 50-300 cm, and a diameter ranging between 8-30 mils or more.

The optional catheter may have one or more lumens for the introduction or delivery of heated fluids (e.g., to induce expansion of shape memory alloys) and may be made of any suitable biocompatible material. Examples of such suitable materials include extruded polymeric materials, such as polyolefins, particularly the polyethylenes, and including other polymers including polyethyleneterephthalate, polyamides, polyesters, polyurethanes, polyvinylchlorides, and the like. Catheters meeting these specifications are well known in the art and are commercially available. Similarly, suitable guidewires for use with such catheters are commercially available. These guides generally comprise an elongate wire having a tapered, wire-wound distal end region adapted to be advanced through a tortuous path. As noted above, in the case of delivery and deployment of stent precursor members within the vasculature, the entry point for delivery may be the femoral artery in the groin. However, other entry points, for example, the neck, are known in the art are also suitable.

Once the delivery device is inserted into the entry point, the stent precursor structure may than be inserted through its lumen. The stent precursor structure may be of a selected desirable length, and may be selected for example, based upon the length of the stent precursor structure required to reach the target site, and the length of stent precursor member required to form a stenting structure of a desirable size. The stent precursor structure (506) is then advanced distally down the lumen of the delivery device, toward the target site (500) as shown in FIG. 16A.

In the variation shown in FIGS. 16A-16D, the method of forming a stenting structure in situ generally comprises the steps of advancing the stent precursor member (508) distally past the target site (500) and releasing it to form a stenting structure (510) at the target site (500). As with any of the methods described herein, angioplasty may optionally be employed prior to the delivery and deployment of the one or more stent precursor members. In this way, the body lumen may be widened prior to the formation of a stenting structure at the target site.

As shown in FIG. 16A, the stent precursor structure (506) is advanced distally past the target site (500). Once the stent precursor member (508) is positioned at a desirable location past target site (500), the delivery device (502) may be pulled proximally as illustrated in FIG. 16B. Proper positioning of the stent precursor member (508) may be accomplished by the use of a radio-opaque material, such as those described in detail above. As the stent precursor member (508) is pulled across a shape forming member, it begins to curve, and the stenting structure begins to form, as shown in FIG. 16C. Once a pre-determined length of stenting structure has been deployed, for example, the stent precursor member (508) is detached from the optional guide member (512) by any of the detachment means discussed above, leaving the stenting structure (510) situated across the target site (500), as shown in FIG. 16D.

For example, when the stent precursor member (508) is electrolytically detachable, the delivery device (502) can be configured to transmit an electrical impulse to detach the stent precursor member (508) from the optional guide member (512). As noted above, the stenting structure (510) can be formed so as to have a variable diameter, such that each turn is in contact with a portion of the target site, helping to maintain the patency of the body lumen.

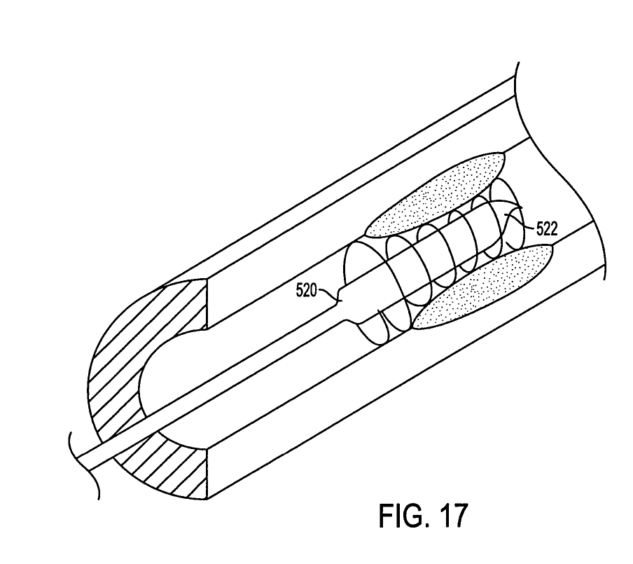

In addition, as shown in FIG. 17, a balloon (520) may optionally be used to ensure the stenting structure (522) is adequately secured to the walls of the target site. In such instances, the balloon is inserted (e.g., on the tip of a balloon catheter) through the lumen of the stenting structure (522), and then expanded (e.g., using pressurized saline, etc.). The balloon expands to contact the walls of stenting structure (522) and provides a gentle force on the stenting structure walls. This in turn helps anchor the stenting structure (522) to the walls of the target site, within the body lumen.

Any number of stent precursor members may be delivered and deployed in using the methods described herein to provide any number of stenting structures. For example, multiple stenting structures may be delivered on top of one other, in order to strengthen the walls of the body lumen. Similarly, multiple stenting structures may be formed adjacent to one another, across the length of a single target site or lesion. In addition, multiple stent precursor structures having multiple stent precursor members may be used to form multiple stenting structures as described above. The stenting structures formed from the multiple stent precursor structures may be formed simultaneously, but need not be. As in the case when a single stent precursor structure is used, the stenting structures formed from multiple stent precursor structures may be delivered to the same or to different target sites.

The stent precursor structures, stenting structures, and delivery systems described herein may also be used as a kit with other implantable devices. Modifications and variations of the device and methods described herein will be apparent to those having skill in the art, and are intended to be within the scope of the claims that follow.

We claim as our invention:

1. A multiple stent precursor assembly comprising: more than one elongated stent precursor member, each stent precursor member at least partially side-by-side to the others to form a bundle, each stent precursor member configured to form stenting structures in situ at a site in a body lumen, and releasably adherent to the bundle.

2. The multiple stent precursor assembly of claim 1 wherein each stent precursor member is configured to be formable into a stent structure in the body lumen by a longitudinal movement with respect to a cooperating delivery element.

3. The multiple stent precursor assembly of claim 1 wherein each stent precursor member is wire-like.

4. The multiple stent precursor assembly of claim 1 where the delivery element comprises at least one guide member adherent to the bundle.

5. The multiple stent precursor assembly of claim 1 where at least one of the stent precursor members is adherent to the bundle substantially along the length of that at least one stent precursor member.

6. The multiple stent precursor assembly of claim 1 where the stent precursor members are configured to release simultaneously from the bundle.

7. The multiple stent precursor assembly of claim 1 where the stent precursor members are configured to release sequentially from the bundle.

8. The multiple stent precursor assembly of claim 7 further comprising one or more clasps releasably holding stent precursor members not to be released from the bundle as another stent precursor member is released sequentially from the bundle.

9. The multiple stent precursor assembly of claim 8 wherein the one or more clasps comprise electrolytically releasable clasps.

10. The multiple stent precursor assembly of claim 1 where the stent precursor members are configured to release a sequence of simultaneously released stent precursor members from the bundle.

11. The multiple stent precursor assembly of claim 1 where at least one stent precursor member is self-forming into the stent structure.

12. The multiple stent precursor assembly of claim 11 where at least one self-forming stent precursor member comprises a super-elastic material.

13. The multiple stent precursor assembly of claim 12 where the super-elastic material is selected from the group consisting of nickel-titanium alloys, copper-zinc alloys, and nickel-aluminum alloys.

14. The multiple stent precursor assembly of claim 12 where the super-elastic material comprises nitinol.

15. The multiple stent precursor assembly of claim 1 where the at least one stent precursor member is self-expanding into the stent structure.

16. The multiple stent precursor assembly of claim 15 where at least one self-expanding stent precursor member comprises a super-elastic material.

17. The multiple stent precursor assembly of claim 16 where the super-elastic material is selected from the group consisting of nickel-titanium alloys, copper-zinc alloys, and nickel-aluminum alloys.

18. The multiple stent precursor assembly of claim 17 where the super-elastic material comprises nitinol.

19. The multiple stent precursor assembly of claim 1 where at least one stent precursor member comprises a plastic material and is of dimensions such that the at least one stent precursor member is plastically deformed upon forming the stent structure.

20. The multiple stent precursor assembly of claim 19 where the plastic material is selected from the group consisting of stainless steels, polyurethanes, ethers, acrylates, olefins, propylene, butenes, butadiene, styrene, and thermoplastic olefin elastomers, polydimethyl siloxane-based polymers, polyethyleneterephthalate, cross-linked polymers, non-cross linked polymers, rayon, cellulose, cellulose derivatives, nitrocellulose, natural rubbers, polyesters, lactides, glycolides, caprolactones and their copolymers and acid derivatives, hydroxybutyrate and polyhydroxyvalerate and their copolymers, polyether esters, anhydrides, hexadecandioic acid, and orthoesters.

21. The multiple stent precursor assembly of claim 19 where the plastic material comprises stainless steel.

22. The multiple stent precursor assembly of claim 19 where the plastic material comprises a polymer.

23. The multiple stent precursor assembly of claim 2 where at least one stent precursor member includes at least a first portion comprising a super-elastic material and at least a second portion comprising a plastic material and having dimensions such that the at least the second portion is plastically deformed upon forming the stent structure.

24. The multiple stent precursor assembly of claim 2 where the at least one stent precursor member further contains a drug.

25. The multiple stent precursor assembly of claim 2 where the at least one stent precursor member further contains a member selected from the group consisting of anti-proliferation agents, anti-inflammatory agents, antibiotics, and immunosuppressants.

26. The multiple stent precursor assembly of claim 2 where the at least one stent precursor member further contains a member selected from the group consisting of paclitaxel, methotrexate, batimastal, doxycycline, tetracycline, rapamycin, actinomycin, dexamethosone, methyl prednisolone, nitroprussides, estrogen, and estradiols.

27. A multiple stent precursor assembly comprising:
a.) at least one guide member, and
b.) more than one elongated stent precursor member, each stent precursor member having a distal end and a proximal end, and (excepting terminal stent precursor members) situated distal end to proximal end, and releasably adherent to at least one of the guide members.

28. The multiple stent precursor assembly of claim 27 wherein each stent precursor member is configured to be formable into a stent structure in the body lumen by a longitudinal movement with respect to a cooperating delivery element.

29. The multiple stent precursor assembly of claim 27 wherein each stent precursor member is wire-like.

30. The multiple stent precursor assembly of claim 27 where at least one of the stent precursor members is adherent to the guide member substantially along the length of that at least one stent precursor member.

31. The multiple stent precursor assembly of claim 27 where the stent precursor members are configured to release sequentially from the guide member.

32. The multiple stent precursor assembly of claim 31 further comprising one or more clasps releasably holding stent precursor members not to be released from the bundle as another stent precursor member is released sequentially from the guide member.

33. The multiple stent precursor assembly of claim 27 where at least one stent precursor member is self-forming into the stent structure.

34. The multiple stent precursor assembly of claim 33 where at least one self-forming stent precursor member comprises a super-elastic material.

35. The multiple stent precursor assembly of claim 34 where the super-elastic material is selected from from the group consisting of nickel-titanium alloys, copper-zinc alloys, and nickel-aluminum alloys.

36. The multiple stent precursor assembly of claim 34 where the super-elastic material comprises nitinol.

37. The multiple stent precursor assembly of claim 27 where at least one stent precursor member comprises a plastic material and is of dimensions such that the at least one stent precursor member is plastically deformed upon forming the stent structure.

38. The multiple stent precursor assembly of claim 27 where the plastic material is selected from the group consisting of stainless steels, polyurethanes, ethers, acrylates, olefins, propylene, butenes, butadiene, styrene, and thermoplastic olefin elastomers, polydimethyl siloxane-based polymers, polyethyleneterephthalate, cross-linked polymers, non-cross linked polymers, rayon, cellulose, cellulose derivatives, nitrocellulose, natural rubbers, polyesters, lactides, glycolides, caprolactones and their copolymers and acid derivatives, hydroxybutyrate and polyhydroxyvalerate and their copolymers, polyether esters, anhydrides, hexadecandioic acid, and orthoesters.

39. The multiple stent precursor assembly of claim 37 where the plastic material comprises a stainless steel.

40. The multiple stent precursor assembly of claim 37 where the plastic material comprises a polymer.

41. The multiple stent precursor assembly of claim 28 where at least one stent precursor member includes at least a first portion comprising a super-elastic material and at least a second portion comprising a plastic material and having dimensions such that the at least the second portion is plastically deformed upon forming the stent structure.

42. The multiple stent precursor assembly of claim 28 where the at least one stent precursor member further contains a drug.

43. The multiple stent precursor assembly of claim 28 where the at least one stent precursor member further contains a member selected from the group consisting of anti-proliferafion agents, anti-inflammatory agents, antibiotics, and immunosuppressants.

44. The multiple stent precursor assembly of claim 28 where the at least one stent precursor member further contains a member selected from the group consisting of paclitaxel, methotrexate, batimastal, doxycycline, tetracycline, rapamycin, actinomycin, dexamethosone, methyl prednisolone, nitroprussides, estrogen, and estradiols.
